SEQUENCE LISTING (15 sequences)

SEQ ID NO: 1 - DNA, 20 bp, Artificial
  synthetic oligonucleotide
taatacgattcactataggg

SEQ ID NO: 2 - DNA, 20 bp, Artificial
  synthetic oligonucleotide
taatacgacccactataggg

SEQ ID NO: 3 - DNA, 20 bp, Artificial
  synthetic oligonucleotide
taatacgacttactataggg

SEQ ID NO: 4 - DNA, 20 bp, Artificial
  synthetic oligonucleotide
taatacgatccactataggg

SEQ ID NO: 5 - DNA, 20 bp, Artificial
  synthetic oligonucleotide
taatacgacctactataggg

SEQ ID NO: 6 - DNA, 20 bp, Artificial
  synthetic oligonucleotide
taatacgatttactataggg

SEQ ID NO: 7 - DNA, 20 bp, Artificial
  synthetic oligonucleotide
taatacgatctactataggg

SEQ ID NO: 8 - DNA, 20 bp, Artificial
  synthetic oligonucleotide
taatacggctcactataggg

SEQ ID NO: 9 - DNA, 20 bp, Artificial
  synthetic oligonucleotide
taatacgactcgctataggg

SEQ ID NO: 10 - DNA, 20 bp, Artificial
  synthetic oligonucleotide
taatacggctcgctataggg

SEQ ID NO: 11 - DNA, 20 bp, Artificial
  synthetic oligonucleotide
taatacgtctcactataggg

SEQ ID NO: 12 - DNA, 20 bp, Artificial
  synthetic oligonucleotide
taatacgcctcactataggg

SEQ ID NO: 13 - DNA, 20 bp, Artificial
  synthetic oligonucleotide
taatacgactctctataggg

SEQ ID NO: 14 - DNA, 20 bp, Artificial
  synthetic oligonucleotide
taatacgactccctataggg

SEQ ID NO: 15 - DNA, 20 bp, Artificial
  synthetic oligonucleotide
taatacgactcactataggg

(19) United States
(12) Patent Application Publication
(10) Pub. No.: US 2020/0277609 A1
(43) Pub. Date: Sep. 3, 2020

(54) METHOD OF OPTIMIZING COMPOUND SYNTHESIS REACTION BASED ON METABOLIC REACTION

(71) Applicant: HITACHI, LTD., Tokyo (JP)

(72) Inventors: Ruolan ZHANG, Tokyo (JP); Hirokazu NISHIDA, Tokyo (JP); Naoko SENDA, Tokyo (JP)

(73) Assignee: HITACHI, LTD.

(21) Appl. No.: 16/787,446

(22) Filed: Feb. 11, 2020

(30) Foreign Application Priority Data
Feb. 28, 2019 (JP) 2019-035873

(51) Int. Cl. C12N 15/113; C12Q 1/6897; G01N 35/00; G01N 21/64

(52) U.S. Cl. CPC C12N 15/1137; C12Q 1/6897; G01N 2021/6439; G01N 21/6428; G01N 35/00584

(57) ABSTRACT

A method of optimizing a synthesis reaction condition for a compound which is synthesized through one or a plurality of reaction steps, the method including: controlling the expression amount of at least one enzyme among one or a plurality of enzymes in the reaction steps by using a wild type and/or a mutant of a promoter; and measuring the synthesis amount of a target compound.

### CROSS-REFERENCE TO RELATED APPLICATION

This application claims priority to Japanese Patent Application No. 2019-035873, filed on Feb. 28, 2019. The disclosure of the prior application is considered part of the disclosure of this application, and is incorporated by reference in its entirety.

### BACKGROUND OF THE INVENTION

### 1. Technical Field

The present invention relates to a method, a kit, and an automation apparatus for optimizing a synthesis reaction condition for a compound which is synthesized through one or a plurality of reaction steps. The present invention further relates to a method of manufacturing a compound under an optimized synthesis reaction condition. The present invention further relates to a new T7 promoter mutant and the use of the same.

### 2. Background Information

“Smart cell industry”, which designs a genome of a microorganism and produces a substance, is in progress. It is expected that the synthesis of protein can be widely applied by using a smart cell. To prepare a smart cell, it is necessary that the concentrations of a plurality of enzymes (proteins) expressed in the cell are controlled to optimize metabolic reaction; however, in the present situation, this technology is not well established.

In the present situation, in general, a flow in which a genome structure is designed (Design), a gene is incorporated into a cell (Build), whether the cell is a cell satisfying specifications or not is checked (Test), and learning is performed (Learn) is repeated until a cell strain of a smart cell is established. However, it takes approximately 6 months to perform DBTL cycles (Design-Build-Test-Learn) running from genome design to the evaluation of the production efficiency of the target substance. Conventional DBTL cycles have been performed mainly in a cell; however, the cost required to establish a cell strain of a smart cell is hundreds of millions of yen per strain, and operations such as transformation and culture take time as long as several years.

A possible solution may be to use a cell-free protein synthesis system that enables high-throughput analysis, and methods using such cell-free synthesis systems have been reported. For example, US 2005/221356 A describes a method of improving the rate of synthesis and the rate of solubilization of a single ligand-binding protein in a cell-free synthesis system. In this method, the synthesis reaction is controlled by adding, as the target ligand, a substance different from those involved in the synthesis reaction, and the protein intended to be synthesized is synthesized in a state of binding to the ligand; hence, the operation of separation from the ligand is needed, and the cost required for the ligand is added.

US 2002/142387 A describes a method in which DNA encoding a protein is fragmented and synthesis is performed in a cell-free system in order to select a domain suitable for the analysis of the stereo-structure of the protein. However, since the DNA encoding the protein is fragmented, this method cannot be applied to a method of synthesizing an enzyme having a sufficient function. Further, the method described in US 2002/142387 A is designed for mass synthesis, and is not designed for the optimization of synthesis reaction.

US 2016/115558 A describes a method for controlling metabolic flux rate in a cell-free system including a set of complicated enzymes, focusing attention on the control of metabolic flux in order to produce a desired product in a pathway of interest, and the metabolic flux rate is controlled by controlling various environmental factors such as enzymes, substrates, and O₂. For example, in the control of an enzyme, the amount of the enzyme is controlled by adding the enzyme or adding a sequence encoding the enzyme; however, the operation is complicated when it is intended to control a plurality of reaction steps simultaneously.

### SUMMARY OF THE INVENTION

When producing a substance in a smart cell, it is necessary to perform multistep reaction by using a plurality of enzymes as catalysts in the metabolic pathway. Further, the amounts of enzymes most suitable for each reaction steps are different; hence, a method of controlling the expression amounts of the enzymes individually is needed. In addition, the enzyme may be influenced by various feedback controls, environmental changes, etc., and the metabolic reaction is further complicated. To increase the production amount of a specified product, it is necessary to measure the changes of enzymes over time. That is, to lessen the time, cost, etc. for constructing a smart cell, an experimental system that can control and measure the expression amounts of a plurality of proteins in wide ranges is needed.

Thus, an object of the present invention is to provide a method and a means for optimizing the production amount of a target compound through the adjustment of the synthesis amount of an enzyme necessary for the synthesis reaction (metabolic pathway) of the compound. Another object of the present invention is to control metabolism by expressing a plurality of enzymes simultaneously and controlling the ratio of each enzyme.

The present inventor conducted extensive studies in order to solve the objects mentioned above, and has found out that compound synthesis reaction can be optimized simply at low cost by, in a cell-free reaction system, using mutants of a promoter having different strengths to control the synthesis amounts of a plurality of enzymes that take part in a plurality of reaction steps; thus, has completed the present invention.

Thus, in one aspect, there is provided a method of optimizing a synthesis reaction condition for a compound synthesizable through one or a plurality of reaction steps,

the method comprising:

controlling the expression amount of at least one enzyme among one or a plurality of enzymes in the reaction steps by using a wild type and/or a mutant of a promoter; and

measuring the synthesis amount of a target compound.

In another aspect, there is provided a method of manufacturing a compound under a synthesis reaction condition optimized by the method described above.

Further, in another aspect, there is provided a kit for optimizing a synthesis reaction condition for a compound which is synthesized through one or a plurality of reaction steps, the kit comprising:

at least one promoter having a sequence selected from the group consisting of:

(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
and
(SEQ ID NO: 14)
TAATACGACTCCCTATAGGG.

Furthermore, in another aspect, there is provided an automation apparatus for synthesizing a compound which is synthesized through one or a plurality of reaction steps,

the apparatus comprising:

a reaction vessel;

an enzyme amount measurement apparatus;

a reagent storage section;

a product amount measurement apparatus; and

a control section,

wherein the expression of one or a plurality of enzymes in the reaction steps under control of a promoter of a wild type and/or a mutant and the synthesis reaction from a reaction substrate to an end product based on the enzyme are performed in the reaction vessel.

Further, in another aspect, there is provided a promoter comprising:

(a) a sequence of:

(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
or
(SEQ ID NO: 14)
TAATACGACTCCCTATAGGG;

or

(b) a sequence including deletion, substitution, or addition of one or a few nucleotides with respect to the sequence of (a) and having promoter activity.

Furthermore, in another aspect, there is provided an expression vector comprising the promoter.

Furthermore, in another aspect, there is provided a cell comprising the expression vector.

Further, in another aspect, there is provided a method of expressing a gene under control of a promoter by using the expression vector or the cell.

According to the present invention, the synthesis reaction of a compound which is synthesized through one or a plurality of reaction steps can be optimized simply, quickly, at low cost, and in minute amounts. Also the manufacturing of a compound in a large scale becomes possible by using a synthesis reaction condition optimized by a method according to the present invention. Thus, the present invention may be useful in fields such as the manufacturing of compounds (particularly proteins) and medicine manufacturing.

### BRIEF DESCRIPTION OF THE DRAWINGS

FIG. 1 shows a reaction outline in which a compound is synthesized through a plurality of reaction steps;

FIG. 2 shows an example of expression vector (plasmid) design and an overview of synthesized enzymes;

FIG. 3 is a schematic diagram showing an operation that measures the synthesis amounts of a plurality of enzymes simultaneously;

FIG. 4 is a schematic diagram showing an operation that analyzes intermediate products and the end product of compound synthesis;

FIG. 5 is a conceptual diagram in which conditions for compound synthesis are optimized by controlling the synthesis amounts of enzymes;

FIG. 6 is a schematic diagram of an automation apparatus having a cell-free protein synthesis system;

FIG. 7 is a diagram showing a binding region of a T7 promoter and T7 RNA polymerase;

FIG. 8 is graphs showing changes over time in fluorescence intensity of green fluorescent proteins (GFPs) corresponding to mutants of a T7 promoter, where WT represents the wild type, and 1 to 14 represent the numbers of the mutants;

FIG. 9 is a schematic diagram of a vector used in Example 1 and a diagram showing the sequences of T7 promoter mutants and the corresponding relative expression intensities; and

FIG. 10 shows a flow chart of an example of a method of optimizing compound synthesis conditions by using mutants of a T7 promoter.

### DETAILED DESCRIPTION OF THE INVENTION

Embodiments will now be described in detail using the drawings. However, the present invention should not be construed as being limited by the subject matter described in the embodiments shown below. A person skilled in the art easily understands that the specific configuration of the present invention can be altered without departing from the idea or gist of the present invention.

The position, size, shape, range, etc. of each configuration shown in the drawings etc. may not show the actual position, size, shape, range, etc., for easier understanding of the invention. Hence, the present invention is not necessarily limited to the position, size, shape, range, etc. disclosed in the drawings etc.

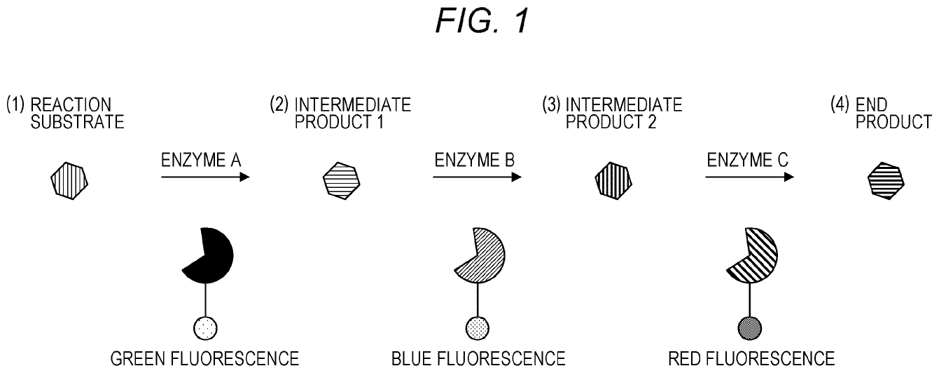

The present invention provides a method and a kit for optimizing a synthesis reaction condition for a compound which is synthesized through one or a plurality of reaction steps, and an apparatus for performing the method by automation. As an example, a reaction outline in which a compound is synthesized through a plurality of reaction steps is shown in FIG. 1. In the synthesis of a compound that is the end product, a reaction substrate is transformed to generate intermediate product 1, intermediate product 2, and the end product by using the catalysts of enzymes A, B, and C, respectively. To enhance the synthesis amounts of the intermediate products and the end product, it may be important to synthesize each enzyme in an appropriate amount.

In one aspect, the present invention provides a method of optimizing a synthesis reaction condition for a compound which is synthesized through one or a plurality of reaction steps,

the method comprising:

controlling the expression amount of at least one enzyme among one or a plurality of enzymes in the reaction steps by using a wild type and/or a mutant of a promoter; and

measuring the synthesis amount of a target compound.

In a method according to the present invention, the expression amount of at least one enzyme among one or a plurality of enzymes in reaction steps of compound synthesis based on metabolic reaction may be controlled by using the wild type and/or a mutant of a promoter. In an embodiment, the expression amount of at least one enzyme may be controlled by using a plurality of the wild type and/or mutant(s) of the promoter. In another embodiment, the expression amounts of a plurality of enzymes may be each controlled by using the wild type and/or a mutant of a promoter. In still another embodiment, the expression amounts of a plurality of enzymes may be each controlled by using a plurality of the wild type and/or mutant(s) of the promoter. What enzyme to control the expression amount of and what wild type/mutant of a promoter to use then may be set in accordance with the kind of the compound intended to be optimized, the number of reaction steps, the scale of reaction, etc., as appropriate. It may be preferable that the expression amount of one or a plurality of enzymes each be controlled by using a plurality of the wild type/mutant(s) of the promoter, and the wild type/mutant of a promoter suitable for each enzyme be finally selected.

According to the present invention, the compound intended to be optimized may not be particularly limited as long as it is a compound which is synthesized through reaction steps based on metabolic reaction, and includes a protein, a glycoprotein, a sugar chain, a lipid, a glycolipid, and the like. The enzyme of which the expression amount is to be controlled may be depending on the kind of the compound, and a person skilled in the art can easily recognize an enzyme necessary for the synthesis of the compound.

The promoter used may not be particularly limited as long as it is one commonly used for gene recombination techniques. The promoter may preferably be a promoter used in a cell-free reaction system; such promoter may include a T7 promoter (used in combination with T7 RNA polymerase), a T3 promoter (used in combination with T3 RNA polymerase), an SP6 promoter (used in combination with SP6 RNA polymerase), a lac promoter, and a tac promoter. According to the present invention, a mutant of a promoter having promoter activity different from the promoter activity of a wild-type promoter, preferably a set of a plurality of mutants, may be prepared, and the expression amount of the enzyme may be controlled at different levels.

In the case where a T7 promoter is used as a promoter, a mutant can be prepared by, for example, introducing a mutation into the T7 RNA polymerase-binding region of the T7 promoter (for example, see Example 1). The mutant of the T7 promoter may not be limited to, but may be, for example, a mutant having a sequence consisting of:

(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
and
(SEQ ID NO: 14)
TAATACGACTCCCTATAGGG.

The wild type of the T7 promoter has the sequence of TAATACGACTCACTATAGGG (SEQ ID NO: 15).

By thus using the wild type and/or a mutant (preferably a plurality of mutants) of a promoter, the kind of the polymerase used for synthesis reaction may be allowed to be one kind corresponding to the kind of the promoter. For example, in the case where T7 promoter mutants are used, the synthesis amount of the enzyme can be controlled at different levels by using T7 RNA polymerase alone. Thus, the change trends of the synthesis amounts of enzymes may be uniform depending on external environmental changes, and the control of synthesis reaction pathway balance may be simpler.

The method according to the present invention may be performed in a cell, but may preferably be performed in a cell-free reaction system. The cell-free reaction system has an advantage in setting optimum conditions for compound synthesis, in terms of quickness, simplicity, low cost, and amount reduction when yielding a recombinant protein in a solution by utilizing the transcription and translation mechanisms of biomolecules extracted from a cell. In the case where a cell-free reaction system is used, a prokaryotic cell-based cell-free reaction system (an Escherichia coli extract or the like) or an eukaryotic cell-based cell-free reaction system (a rabbit reticulocyte lysate, an insect cell extract, a wheat germ extract, or the like) may be used in accordance with the compound intended to be optimized. On the other hand, in the case where a cell is used, a host cell generally used for gene recombination or protein production, such as a bacterium (Escherichia coli, Bacillus subtilis, or the like), a yeast, a plant cell, an animal cell (a COS cell, a CHO cell, or the like), or an insect cell, may be used in accordance with the compound intended to be optimized.

The expression of an enzyme controlled by a mutant of a promoter can be performed by a method known in this technical field. Specifically, an expression vector can be prepared by linking (inserting), to an adequate vector, a promoter (wild type/mutant) and a gene encoding an enzyme. The vector used in the present invention may not be particularly limited as long as it is one that can be used in a cell-free reaction system or one that can be replicated in a host cell; and can be selected by a person skilled in the art, as appropriate. A cloning vector suitable for such a use may be commercially available, and may be included in a kit for a cell-free reaction system. Alternatively, in a cell-free reaction system, transcription and translation can be performed also by using linear DNA; thus, linear DNA for expression can be prepared by simply linking (inserting) a promoter (wild type/mutant) and a gene encoding an enzyme.

To insert, into a vector, a promoter and a gene (DNA) encoding an enzyme, a purified DNA may be cut with adequate restriction enzymes and then the promoter and the gene may be inserted into restriction enzyme sites or a multicloning site of adequate vector DNA to ligate them to the vector, for example. As well as the sequences of a promoter and a gene, a cis element such as an enhancer, a splicing signal, a poly(A) addition signal, a selective marker, a ribosome-binding sequence (the SD sequence), a terminator sequence, etc. may be linked to the vector or the linear DNA for expression described above, as desired.

In the case where a cell-free reaction system is used, a prepared vector or prepared linear DNA may be introduced into a cell-free reaction system together with a polymerase, a reaction substrate, etc., and thereby an enzyme may be expressed under the control of the promoter.

In the case where a cell is used, a prepared vector may be introduced into a host cell so that a coded enzyme can be expressed. The method for introducing an expression vector into a host cell may not be particularly limited as long as it is a method known in this technical field. Examples include a method using a calcium ion, the electroporation method, the spheroplast method, the lipofection method, etc.

By the steps described above, the expression amount of an enzyme may be controlled on the basis of the promoter activity strength of a promoter mutant, which is different from the promoter activity strength of the wild type.

The method according to the present invention may be one further including a step of measuring the controlled expression amount of an enzyme. An object of the present invention is to increase the synthesis amount of a target compound that is the end product; the optimum amount of an enzyme and the optimum ratio of one or a plurality of enzymes can be understood by measuring the expression amount of an enzyme contributing to each reaction step of synthesis. The method for measuring the expression amount of an enzyme may not be particularly limited, and a method known in this technical field, such as a mass spectrometer (MS) or high-performance liquid chromatography (HPLC), may be used.

In an embodiment, the step of measuring the expression amount may be conducted by using a tag protein. Here, the tag protein refers to any protein that is placed under the control of the same promoter (wild type/mutant) as the promoter for the enzyme and of which the measurement of the amount allows the expression amount of the enzyme to be found. The tag protein may preferably be one of which the amount can be measured simply; for example, at least one of a fluorescent protein, a luminescent protein, a chromoprotein, and the like (parts of these are possible as long as each of them is one that emits a measurable signal) may be used. The tag protein and the enzyme may be expressed as a fusion protein or expressed as independently expressed proteins as long as both are under the control of the same promoter. In the case where the expression amounts of a plurality of enzymes are controlled, a plurality of fluorescent proteins that emit different kinds of fluorescence may be used for the plurality of enzymes, respectively.

The measurement of the amount of the tag protein may vary with the kind of the tag protein used, and a person skilled in the art can select an adequate method. For example, in the case where a fluorescent protein is used, the color and intensity of fluorescence can be detected by a spectrophotometer, a fluorescence microscope, or the like. The measurement apparatus for measuring such a tag protein may also function as a place for the expression of an enzyme or the synthesis reaction described above. Thus, the method according to the present invention can be performed in a minute-amount reaction system.

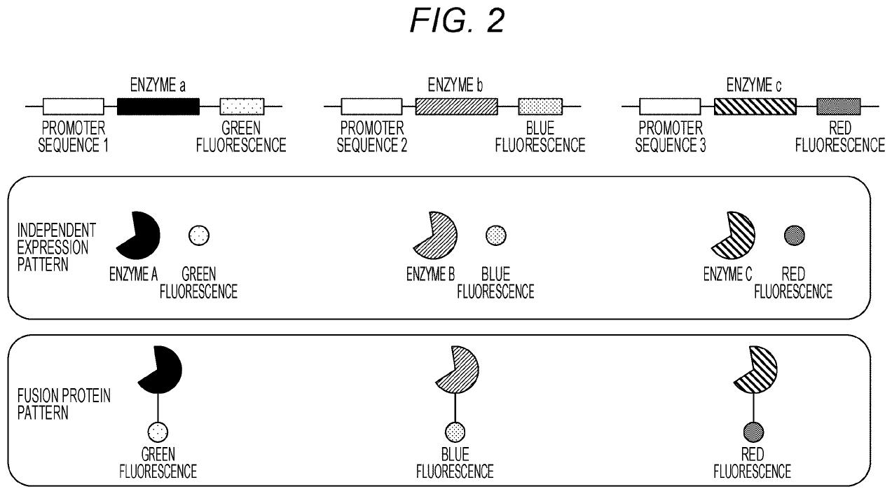

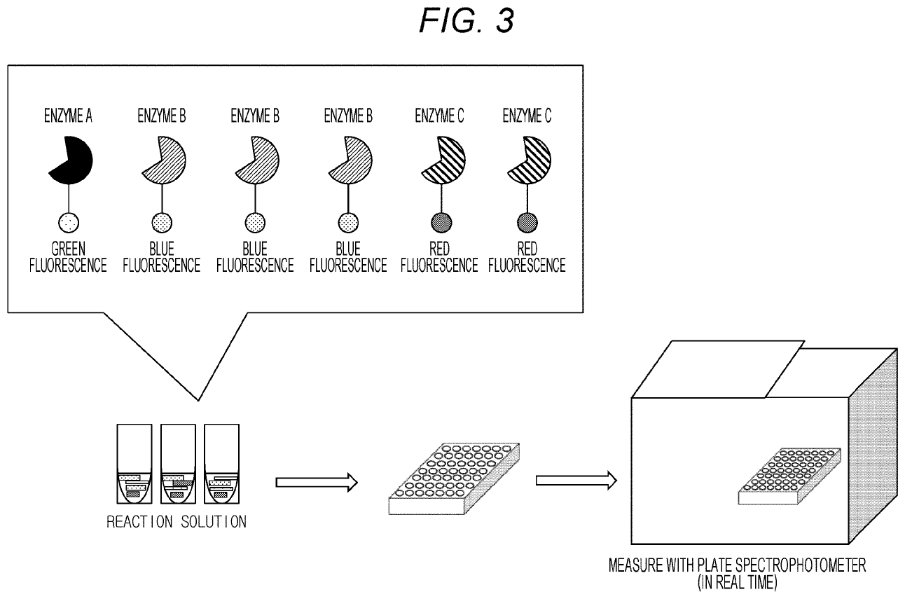

An example of expression vector (plasmid) design and an overview of synthesized enzymes in the case where a plurality of fluorescent proteins are used as tag proteins are shown in FIG. 2. As shown in FIG. 2, the expressions of Enzyme a, Enzyme b, and Enzyme c may be controlled by different promoter mutants (Promoter sequence 1, Promoter sequence 2, and Promoter sequence 3, respectively). As tag proteins, genes that encode fluorescent proteins with different colors (green, blue, and red) or parts of them correspond to genes that encode Enzyme a, Enzyme b, and Enzyme c, respectively, and the expression of each pair collectively may be controlled by the respective promoter mutant. Specifically, the sequences of promoter mutants having different strengths of promoter activity and sequences that code enzymes and tag proteins may be linked together (the upper side of FIG. 2). The enzyme and the fluorescent protein may be ones expressed independently of each other, or may be ones expressed as a fusion protein (the lower side of FIG. 2). In both cases, the expression amount of the enzyme can be calculated on the basis of the amount of the tag protein. In the case where, as shown in FIG. 2, different colors of fluorescence may be used for a plurality of enzymes to be controlled, respectively, the expression amounts of a plurality of tag proteins (that is, the plurality of enzymes) can be measured simultaneously, as shown in FIG. 3. Thus, the optimum amount of each enzyme can be checked without performing a sampling procedure, which has been necessary in a conventional method.

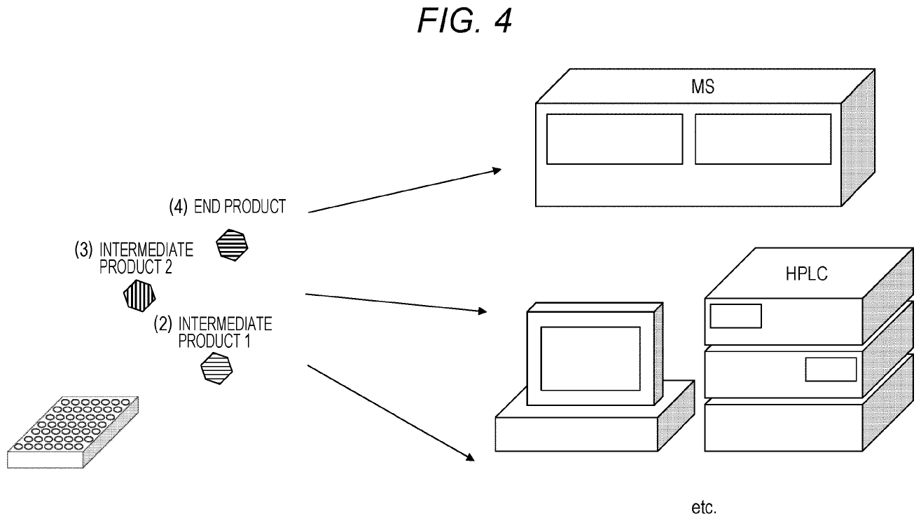

According to the method of the present invention, the synthesis amount of a target compound (the end product) may be measured. Further, the synthesis amount of an intermediate product of each reaction step may be optionally measured. Such measurement of an intermediate product and a compound (the end product) may vary in accordance with the kinds of the intermediate product and the compound (the end product) of interest, and a person skilled in the art can use an adequate method. For example, the synthesis amount of an intermediate product and/or a compound (the end product) may be measured or monitored by using a mass spectrometer (MS), high-performance liquid chromatography (HPLC), a liquid chromatography mass spectrometer (LC/MS), an NMR analyzer, a two-dimensional electrophoresis apparatus, etc. (FIG. 4).

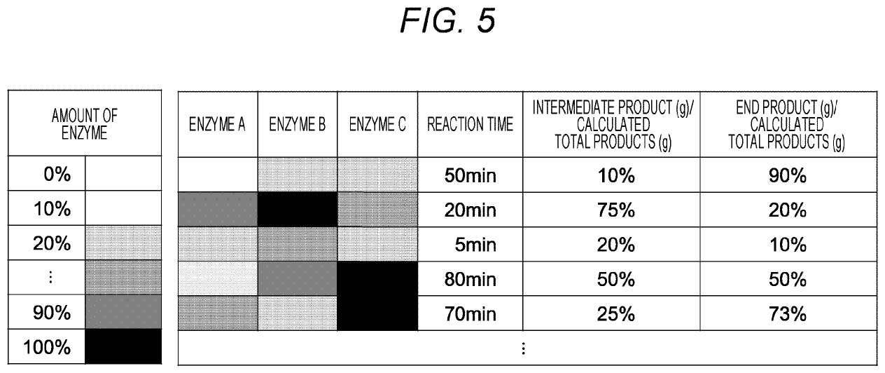

FIG. 5 shows a conceptual diagram in which conditions for compound synthesis are optimized by controlling the synthesis amounts of enzymes. Specifically, an example case where an intermediate product and the end product are synthesized by a metabolic pathway including three steps of catalytic reaction is given. The expression amount of protein in the case where a wild-type promoter sequence may be used as a standard (100%). The amounts of the intermediate product and the end product generated may vary when the synthesis amounts of Enzyme A, Enzyme B, and Enzyme C are controlled (in FIG. 5, 10 to 100%). For example, it can be seen that, when it is attempted to construct a cell strain that equalizes the amount of the intermediate product and the amount of the end product, the synthesis amount of Enzyme A may be set to 10%, the synthesis amount of Enzyme B to 90%, and the synthesis amount of Enzyme C to 100%. Further, for example, it can be seen that, when it is attempted to construct a cell strain that maximizes the amount of the end product, the synthesis amount of Enzyme A may be set to 10%, the synthesis amount of Enzyme B to 20%, and the synthesis amount of Enzyme C to 20%. The control of the synthesis amounts of enzymes and the optimization of the amounts of products can be achieved simply and quickly by using promoter mutants corresponding to relative expression amounts. In this way, the optimum amounts of enzymes (the enzyme ratio) may be obtained as optimized synthesis reaction conditions. Further, optimum reaction time may be optionally determined.

Thus, in another aspect, the present invention provides a method of manufacturing a compound under a synthesis reaction condition optimized by the method described above. The manufacturing of a compound may be performed either in a cell or in a cell-free reaction system as long as optimized synthesis reaction condition is used. In terms of stable, large-scale compound manufacturing, it may be preferable to manufacture a compound in a cell. Thus, a cell strain (a smart cell) in which compound synthesis is optimized can be bred by reproducing, in a cell, a synthesis reaction condition optimized by the method described above. According to the present invention, after an optimum synthesis reaction condition is determined quickly, simply, and in minute amounts, a compound can be synthesized in a large scale by using the optimized synthesis reaction condition.

The method according to the present invention is a control technique widely applicable to the synthesis of low molecular compounds. The range of synthesizable enzymes may be wide, and sampling is not needed. The synthesis amounts of enzymes may be measured on a real time basis by using a wide variety of tag proteins (fluorescent reporters). Since the number of variables of the present invention is small, it has become possible to control a synthesis reaction more easily and quickly than in a conventional technique. The construction of a model and the estimation of the amount of a product may be enabled by using existing data Also application to metabolic control and analysis in a cell is possible.

The method according to the present invention can be performed more easily and simply by using a kit including at least one T7 promoter mutant. That is, in another aspect, the present invention provides a kit for optimizing a synthesis reaction condition for a compound which is synthesized through one or a plurality of reaction steps, the kit comprising:

at least one promoter having a sequence selected from the group consisting of:

(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
and
(SEQ ID NO: 14)
TAATACGACTCCCTATAGGG.

The kit according to the present invention may include a wild-type T7 promoter in addition to a T7 promoter mutant. These promoters may be in an expression vector. For example, in an expression vector, a T7 promoter mutant or a wild-type T7 promoter mentioned above may be inserted, and as necessary also a multicloning site, a gene that codes a tag protein, a terminator sequence, etc. may be inserted. Thus, a synthesis reaction condition for a compound of which synthesis reaction condition is intended to be optimized can be simply optimized by merely inserting a gene that codes the compound into the expression vector.

The kit according to the present invention may further include other components that are needed when performing the optimization of synthesis reaction condition for a compound, such as T7 RNA polymerase and a substrate. The kit may further include an instruction in which a procedure and a protocol for performing the optimization of synthesis reaction condition for a compound are written.

Further, in another aspect, the present invention provides an automation apparatus for synthesizing a compound which is synthesized through one or a plurality of reaction steps,

the apparatus comprising:

a reaction vessel;

an enzyme amount measurement apparatus;

a reagent storage section;

a product amount measurement apparatus; and

a control section,

wherein the expression of one or a plurality of enzymes in the reaction steps under control of a promoter of a wild type and/or a mutant and synthesis reaction from a reaction substrate to an end product based on the enzyme are performed in the reaction vessel.

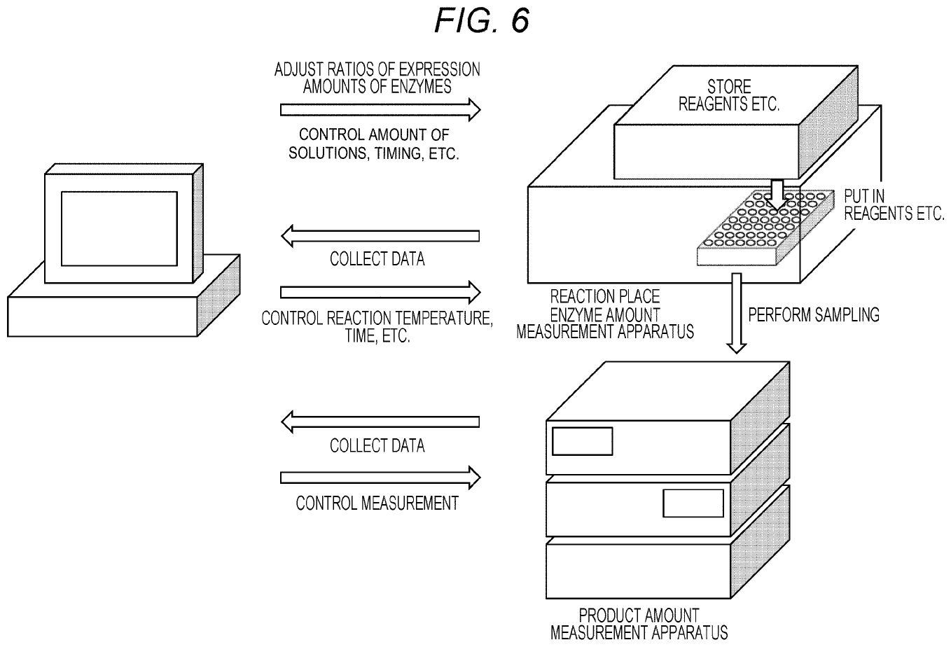

FIG. 6 shows a schematic diagram of an automation apparatus having a cell-free protein synthesis system. The automation apparatus may be composed of a reaction vessel, an enzyme amount measurement apparatus, a reagent storage section, a product amount measurement apparatus, and a control section.

In the reaction vessel, the expression of one or a plurality of enzymes in reaction steps and synthesis reaction from a reaction substrate to the end product based on one or a plurality of enzymes may be performed. As the reaction vessel, any vessel known in this technical field may be used, and an adequate size and an adequate material may be selected in accordance with the purpose. For example, in the case where reaction is performed in minute amounts, a plate having wells, such as a 96-well plate, may be used. Alternatively, in the case where reaction is performed in a cell, a vessel suitable for cell culture may be employed as the reaction vessel. The reaction vessel may be connected to a temperature control section, a pH control section, etc. for setting conditions most suitable for reaction performed in the reaction vessel.

In the reagent storage section, reagents necessary for reaction performed in the reaction vessel, such as a vector or linear DNA for expressing an enzyme under the control of a promoter of the wild type and/or a mutant, a polymerase, a substrate, a starting material, a buffer, and a culture medium, may be stored.

The enzyme amount measurement apparatus may not be particularly limited as long as it is an apparatus capable of measuring the amount of an enzyme. For example, a mass spectrometer (MS), a high-performance liquid chromatography (HPLC) spectrophotometer, a fluorescence microscope, etc. may be used. The measurement of the amount of an enzyme may preferably be performed in the reaction vessel without performing the sampling of reaction solution from the reaction vessel.

The product amount measurement apparatus may not be particularly limited as long as it is an apparatus capable of measuring the amounts of a compound (the end product) and an intermediate product synthesized in the reaction vessel. For example, a mass spectrometer (MS), a high-performance liquid chromatography (HPLC) apparatus, a liquid chromatography mass spectrometer (LC/MS), an NMR analyzer, a two-dimensional electrophoresis apparatus, etc. may be used.

The control section may be configured to control each component connected directly or via wireless; for example, control at least one of the adjustment of the ratio of the expression amount of an enzyme in the reaction vessel, the control of the amount, timing, etc. of a reagent put in from the reagent storage section into the reaction vessel, the collection of data from the enzyme amount measurement apparatus, the control of the reaction temperature, time, etc. in the reaction vessel, the collection of data from the product amount measurement apparatus, and the control of the measurement of the amount of a product obtained from the reaction vessel. For example, the control section may be configured so as to control the temperature and time of reaction in the reaction vessel and the amount and timing of a reagent put into the reaction vessel on the basis of output data collected from the enzyme amount measurement apparatus mentioned above. As the control section, any control means known in this technical field may be used; for example, a computer may be used.

The automation apparatus may include an output apparatus, or may be connected to an external output apparatus. The output apparatus may be any output apparatus known in this technical field, and examples include an image and/or data display apparatus, an alarm apparatus, a printer, etc. The automation apparatus may further include a memory section that stores collected data.

In another aspect, the present invention provides a promoter comprising:

(a) a sequence of:

(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
or
(SEQ ID NO: 14)
TAATACGACTCCCTATAGGG;

or

(b) a sequence including deletion, substitution, or addition of one or a few nucleotides with respect to the sequence of (a) and having promoter activity.

A promoter including the sequence of any of SEQ ID NOs: 1 to 14 is a mutant of a wild-type T7 promoter; in Example 1, all have been found to possess a promoter activity of less than 1.5% to 126%. Hence, gene expression at a desired level can be performed by selecting a promoter having promoter activity according to the purpose. For example, in the case where a strong promoter is desired, a promoter having the sequence of SEQ ID NO: 9 or SEQ ID NO: 12, which has high promoter activity, may be used; on the other hand, in the case where a weak promoter is desired (in the case where the inhibition or suppression of gene expression is desired), a promoter having the sequence of any of SEQ ID NOs: 4 to 7 and SEQ ID NO: 10, which has low promoter activity, may be used. Here, the promoter activity refers to, when a gene is operably linked downstream of a promoter and is introduced into to a host, having the capability and function of transcribing the gene in the host or outside the host. The sequence of (b) mentioned above, that is, a sequence including the deletion, substitution, or addition of one or a few nucleotides with respect to any of SEQ ID NOs: 1 to 14 has only to have promoter activity, and refers to maintaining enough promoter activity to allow almost similar use under the same conditions as conditions under which a promoter consisting of the respective sequence of any of SEQ ID NOs: 1 to 14 functions. For example, the sequence of (b) is a sequence that maintains an activity of approximately 0.01 to 100 times, preferably approximately 0.5 to 20 times, and more preferably approximately 0.5 to 2 times the activity of a promoter consisting of the respective sequence of any of SEQ ID NOs: 1 to 14.

Such promoter can be prepared in conformity with a method known in this technical field by referring to the sequence of any of SEQ ID NOs: 1 to 14. For example, an oligonucleotide having a promoter sequence can be simply synthesized by chemical synthesis. Alternatively, a technology in which deletion, substitution, or addition is artificially performed from the sequence of a wild-type T7 promoter (SEQ ID NO: 15) or the sequence of a mutant (any of SEQ ID NOs: 1 to 14), such as the site-specific mutagenesis method, may be used, and thereby a mutant with a different sequence can be prepared while promoter activity is maintained.

Whether a mutant of a promoter obtained in the above manner has activity as a promoter or not can be checked by the following technique. That is, the promoter activity of a promoter obtained in the above manner can be checked by preparing a vector in which preferably a reporter gene, such as a luciferase gene (LUC), a chloramphenicol acetyl transferase gene (CAT), and a β-galactosidase gene (GAL), is linked to a region downstream of the promoter, adding the vector to a cell-free reaction system or introducing the vector into a host cell, and then measuring the expression of the reporter gene.

An expression vector according to the present invention can be obtained by linking a promoter according to the present invention to an adequate vector. A gene may be operably linked to the expression vector. The vector for inserting a promoter (and a gene) may not be particularly limited as long as it is a vector commonly used in this technical field, and examples include a plasmid, a viral vector, a bacteriophage, an artificial chromosome vector, and the like. To insert a promoter (and a gene) into a vector, a purified DNA may be cut with adequate restriction enzymes and then inserted into a restriction enzyme site or a multicloning site of adequate vector DNA to be linked to the vector, or the like.

A promoter according to the present invention needs to be operably inserted into a vector such that the function of a gene is exerted. “Operably” means that the gene and the promoter are linked together and are incorporated into the vector such that the gene is expressed under the control of the promoter. In a preferred embodiment, a gene and a terminator (a T7 terminator), and as desired a cis-element such as an enhancer, a splicing signal, a poly(A) addition signal, a selective marker, a ribosome-binding sequence (the SD sequence), etc. may be linked, as well as a promoter, to an expression vector according to the present invention.

A cell according to the present invention can be obtained by introducing an expression vector according to the present invention into a host cell such that a gene can be expressed under the control of a promoter. Here, the host cell may not be particularly limited as long as it is one in which a gene can be expressed under the control of a promoter according to the present invention. For example, a bacterium (Escherichia coli or the like), a yeast, an animal cell, a plant cell, an insect cell, or the like may be used as the host cell. The method for introducing an expression vector into a host cell may not be particularly limited as long as it is a method known in this technical field. Examples include a method using a calcium ion, the electroporation method, the spheroplast method, the lipofection method, a method using Agrobacterium, etc.

The present invention further provides a method of expressing a gene under the control of a promoter according to the present invention by using an expression vector according to the present invention or a cell according to the present invention. For example, an expression vector according to the present invention may be added to a cell-free reaction system, or a cell according to the present invention may be cultured, and thereby a gene can be expressed under the control of a promoter on the expression vector. All techniques like these are operations commonly performed in this technical field, and a person skilled in the art can perform the method according to the present invention under adequate conditions in accordance with the cell-free reaction system used, the kind of the cell, etc.

In the following, Examples are illustrated, and the present invention is specifically described; however, these Examples are provided merely for description of the present invention, and do not limit or narrow the scope of the invention disclosed in the present application.

### EXAMPLES

### Example 1

The present Example describes an example in which the nucleotide sequence of the T7 RNA polymerase-binding region of a T7 promoter is changed and thereby the relative expression amount is changed to 1 to 126% in the case where the expression amount of protein under the control of the wild-type T7 promoter sequence is used as a standard.

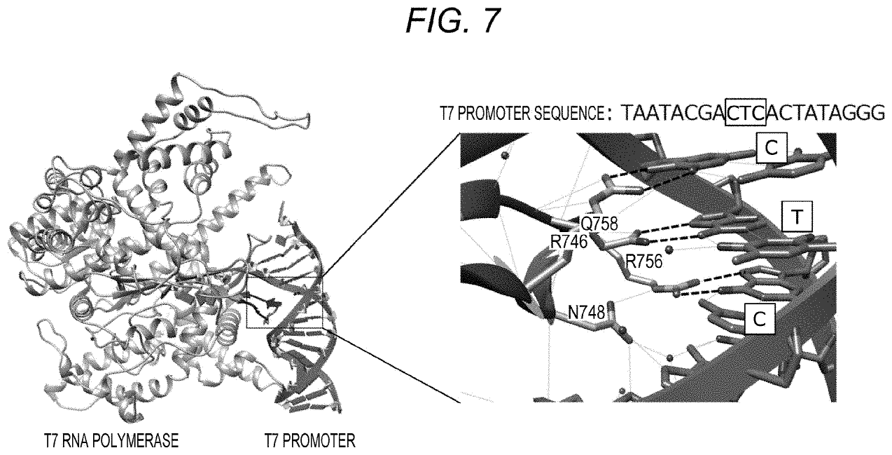

A co-crystal structure of a T7 promoter and T7 RNA polymerase used in the present Example is shown in FIG. 7. The sequence from the first base, T, to the 13th base, C, of the T7 promoter sequence is referred to as a T7 polymerase-binding region, and influences binding affinity with the polymerase and the expression amount of a protein. Herein, the nucleotide sequence site complementary to three bases of CTC (the 9th base to the 11th base) of the T7 promoter creates hydrogen bonds with hydrophilic amino acids of the RNA polymerase; therefore, it was presumed that the nucleotide sequence of CTC of the T7 promoter would particularly influence transcription efficiency. Hence, a plurality of mutants in which nucleotide substitution was performed centering on the nucleotide sequence of CTC of the T7 promoter were designed.

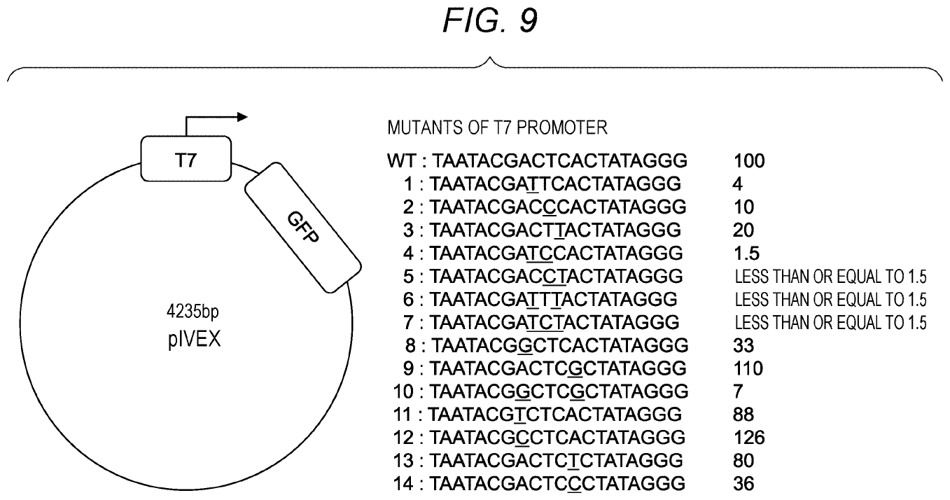

Specifically, a vector having a mutant of a T7 promoter designed as a promoter and having a green fluorescent protein (GFP) as a reporter protein will now be described. For a plasmid, pIVEX2.3 GFPwt (attached to RTS 100 E. coli HY Kit; biotechrabbit GmbH) was used as a backbone (the left side of FIG. 9). Point mutation was introduced into the T7 promoter sequence on the basis of a designed mutation introduction primer by using QuikChange Site-Directed Mutagenesis Kit (Agilent Technologies Japan, Ltd.). In the case where two or more nucleotides were substituted as compared to the wild-type sequence, the mutant was prepared by performing point mutation based on QuikChange multiple times. The sequences of specific mutants are as shown in the right side of FIG. 9, where the underline indicates the mutated site.

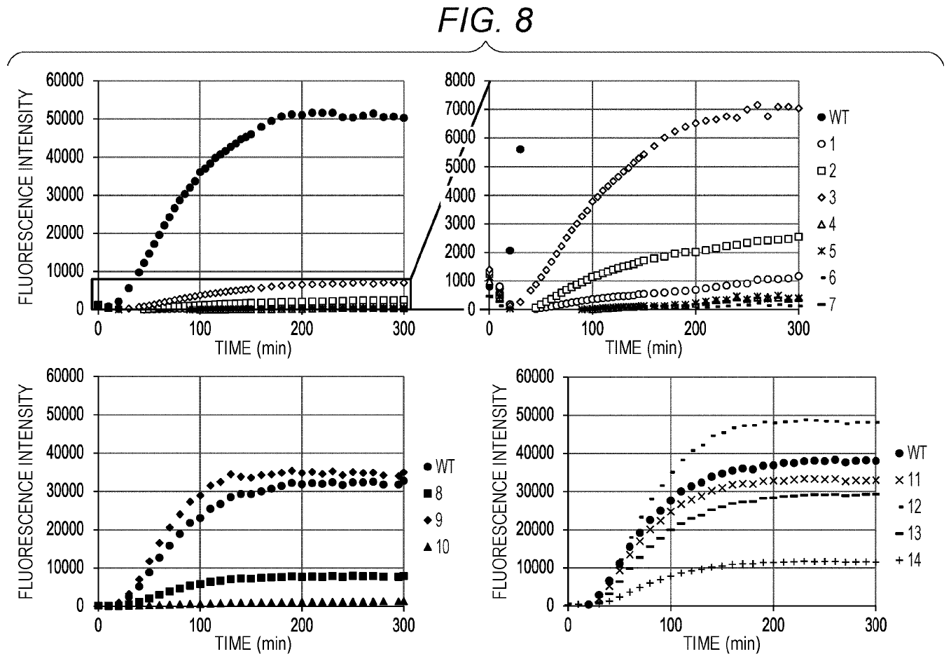

Next, a plurality of designed plasmids were used to evaluate the expression amounts of GFPs. For the evaluation, a fluorescent protein was synthesized by using 20 μL of a cell-free protein synthesis reaction solution (“Cell-free Quick kit”, sold by Taiyo Nippon Sanso Corporation), and the fluorescence intensity of the GFP was measured on a real time using a plate spectrophotometer. The changes over time in GFP fluorescence intensity in reaction solutions each after a plasmid containing a mutant of the promoter sequence was put in are shown in FIG. 8. FIG. 8 shows changes over time in fluorescence intensity of the wild type and mutants 1 to 14.

The right side of FIG. 9 and the following show the calculated expression amount ratios of the GFPs in the case where T7 promoter mutants were used. The fluorescence intensity ratio at the time point when the fluorescence intensity had reached plateau was calculated. For a total of 14 promoter mutants in which mutation was introduced into the 8th base to the 12th base of the T7 promoter, the expression amounts in the case where the expression amount of protein under the control of the wild-type T7 promoter sequence (TAATACGACTCACTATAGGG; SEQ ID NO: 15) is set to 100% are as follows:

Mutant 1 (TAATACGATTCACTATAGGG: SEQ ID NO: 1), expression amount: 4%

Mutant 2 (TAATACGACCCACTATAGGG: SEQ ID NO: 2), expression amount: 10%

Mutant 3 (TAATACGACTTACTATAGGG: SEQ ID NO: 3), expression amount: 20%

Mutant 4 (TAATACGATCCACTATAGGG: SEQ ID NO: 4), expression amount: less than or equal to 1.5%

Mutant 5 (TAATACGACCTACTATAGGG: SEQ ID NO: 5), expression amount: less than or equal to 1.5%

Mutant 6 (TAATACGATTTACTATAGGG: SEQ ID NO: 6), expression amount: less than or equal to 1.5%

Mutant 7 (TAATACGATCTACTATAGGG: SEQ ID NO: 7), expression amount: less than or equal to 1.5%

Mutant 8 (TAATACGGCTCACTATAGGG: SEQ ID NO: 8), expression amount: 33%

Mutant 9 (TAATACGACTCGCTATAGGG: SEQ ID NO: 9), expression amount: 110%

Mutant 10 (TAATACGGCTCGCTATAGGG: SEQ ID NO: 10), expression amount: 7%

Mutant 11 (TAATACGTCTCACTATAGGG: SEQ ID NO: 11), expression amount: 88%

Mutant 12 (TAATACGCCTCACTATAGGG: SEQ ID NO: 12), expression amount: 126%

Mutant 13 (TAATACGACTCTCTATAGGG: SEQ ID NO: 13), expression amount: 80%

Mutant 14 (TAATACGACTCCCTATAGGG: SEQ ID NO: 14), expression amount: 36%

The mutants of the T7 promoter prepared in the above manner allow the same polymerase, that is, T7 RNA polymerase to bind thereto. Hence, a plurality of genes to which different T7 promoter mutants are respectively linked can be transcribed and expressed by using the same polymerase, that is, under the same reaction conditions; thus, the expression of a plurality of genes can be controlled by a simple operation.

### Example 2

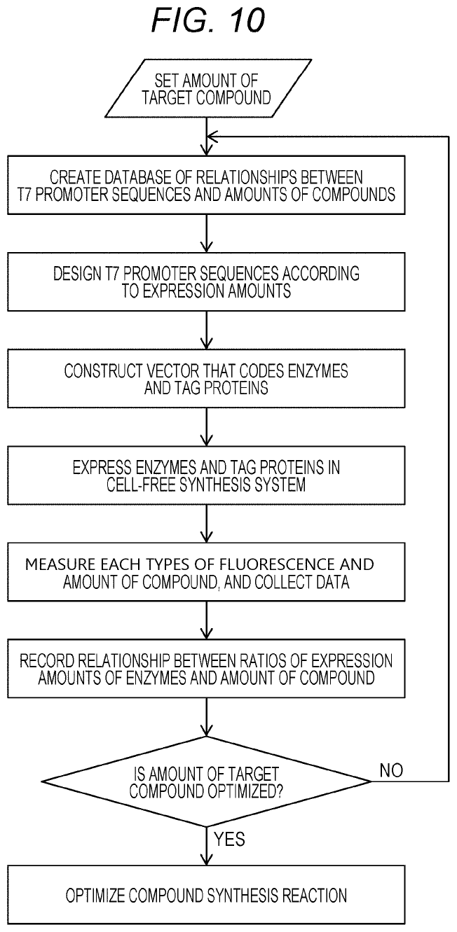

In the present Example, an example of a method of optimizing a reaction condition for compound synthesis by using the mutants of a T7 promoter prepared as described in Example 1 is described using the flow chart of FIG. 10.

FIG. 10 shows an overall concept of the present invention. First, there is a database of the relationships between T7 promoter sequences and the amounts of compounds. The synthesis amount of a target compound has been set through multistep metabolic reaction. To achieve this synthesis amount of the compound, an adequate T7 promoter sequence for each enzyme used for the multistep metabolic reaction is searched for from the database. After T7 promoter sequences according to the expression amounts of the enzymes are designed, sequences that code the enzymes and tag proteins are inserted into a plasmid that contains the T7 promoter sequences mentioned above; thus, a vector is constructed. The constructed vector is put into a cell-free synthesis system, and the enzymes and the tag proteins are expressed. After that, the amounts of the tag proteins and the amount of the compound in the cell-free synthesis system are measured, and data are collected. The expression amount of the enzyme is obtained from the amount of the tag protein. The relationship between the ratios of the expression amounts of the enzymes and the amount of the compound is recorded. At this time, whether the amount of the target compound is optimized or not is assessed.

In the case where the amount of the target compound is optimized, the target compound is produced by using the vector, which can express each enzyme in an appropriate expression amount. In the case where the amount of the target compound is not optimized, the procedure returns to the beginning of the cycle, and the ratio of each enzyme is designed again. At this time, on the basis of the newly obtained experimental data, the database of the relationships between T7 promoter sequences and the amounts of compounds is updated so as to obtain a more appropriate solution. The updating of the database is not limited to the case where the amount of the target compound is not optimized, and is performed as necessary. In this way, the optimization of compound synthesis reaction is performed.

What is claimed is:

1. A method of optimizing a synthesis reaction condition for a compound which is synthesized through one or a plurality of reaction steps,
the method comprising:
controlling the expression amount of at least one enzyme among one or a plurality of enzymes in the reaction steps by using a wild type and/or a mutant of a promoter; and
measuring the synthesis amount of a target compound.

2. The method according to claim 1, wherein the controlling the expression amount of the enzyme comprises controlling the expression amount of at least one enzyme by using a plurality of the wild type and/or mutant of the promoter.

3. The method according to claim 1, wherein the controlling the expression amount of the enzyme comprises controlling the expression amounts of a plurality of enzymes by using the wild type and/or mutant of the promoter.

4. The method according to claim 1, wherein the controlling the expression amount of the enzyme comprises controlling the expression amounts of a plurality of enzymes by individually using a plurality of the wild type and/or mutant of the promoter.

5. The method according to claim 1, wherein the promoter is a T7 promoter.

6. The method according to claim 5, wherein the mutant of the T7 promoter is a mutant having a sequence selected from the group consisting of:
(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
and
(SEQ ID NO: 14)
TAATACGACTCCCTATAGGG.

7. The method according to claim 1, further comprising: measuring the controlled expression amount of the enzyme.

8. The method according to claim 7, wherein the measuring the expression amount is conducted by using a tag protein.

9. The method according to claim 8, wherein the tag protein comprises at least one selected from the group consisting of a fluorescent protein and a luminescent protein.

10. The method according to claim 9, wherein the fluorescent protein comprises a plurality of fluorescent proteins that emit different kinds of fluorescence for a plurality of enzymes.

11. The method according to claim 1, further comprising: measuring the synthesis amount of at least one intermediate product of the reaction steps.

12. The method according to claim 1, wherein the method is performed in a cell-free reaction system.

13. The method according to claim 1, wherein the compound comprises at least one selected from the group consisting of a protein, a glycoprotein, a sugar chain, a lipid, and a glycolipid.

14. A method of manufacturing a compound under a synthesis reaction condition optimized by the method according to claim 1.

15. The method according to claim 14, wherein the compound is manufactured in a cell under the synthesis reaction condition.

16. A kit for optimizing a synthesis reaction condition for a compound which is synthesized through one or a plurality of reaction steps, the kit comprising:
at least one kind of promoter including a sequence selected from the group consisting of
(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
and
(SEQ ID NO: 14)
TAATACGACTCCCTATAGGG.

17. The kit according to claim 16, wherein the promoter is in an expression vector.

18. The kit according to claim 16, further comprising: T7 RNA polymerase.

19. An automation apparatus for synthesizing a compound which is synthesized through one or a plurality of reaction steps,
the apparatus comprising:
a reaction vessel;
an enzyme amount measurement apparatus;
a reagent storage section;
a product amount measurement apparatus; and
a control section,
wherein the expression of one or a plurality of enzymes in the reaction steps under control of a promoter of a wild type and/or a mutant and synthesis reaction from a reaction substrate to an end product based on the enzyme are performed in the reaction vessel.

20. A promoter comprising:
(a) a sequence of:
(SEQ ID NO: 1)
TAATACGATTCACTATAGGG,
(SEQ ID NO: 2)
TAATACGACCCACTATAGGG,
(SEQ ID NO: 3)
TAATACGACTTACTATAGGG,
(SEQ ID NO: 4)
TAATACGATCCACTATAGGG,
(SEQ ID NO: 5)
TAATACGACCTACTATAGGG,
(SEQ ID NO: 6)
TAATACGATTTACTATAGGG,
(SEQ ID NO: 7)
TAATACGATCTACTATAGGG,
(SEQ ID NO: 8)
TAATACGGCTCACTATAGGG,
(SEQ ID NO: 9)
TAATACGACTCGCTATAGGG,
(SEQ ID NO: 10)
TAATACGGCTCGCTATAGGG,
(SEQ ID NO: 11)
TAATACGTCTCACTATAGGG,
(SEQ ID NO: 12)
TAATACGCCTCACTATAGGG,
(SEQ ID NO: 13)
TAATACGACTCTCTATAGGG,
or
(SEQ ID NO: 14)
TAATACGACTCCCTATAGGG;
or
(b) a sequence comprising deletion, substitution, or addition of a few nucleotides with respect to the sequence of (a) and having promoter activity.

21. An expression vector comprising the promoter according to claim 20.

22. The expression vector according to claim 21, wherein a gene is functionally linked.

23. A cell comprising the expression vector according to claim 22.

24. A method of expressing a gene under control of a promoter by using the expression vector according to claim 22.
